(19) United States
(12) Patent Application Publication
(10) Pub. No.: US 2006/0194268 A1
(43) Pub. Date: Aug. 31, 2006

(54) Method for measuring spinorphin

(76) Inventors: Tadahiko Hazato, Asaka-shi (JP); Yukio Yamamoto, Nagareyama-shi (JP); Yoshiro Yamamura, Osaka (JP)

Correspondence Address: WENDEROTH, LIND & PONACK, L.L.P., 2033 K STREET N. W., SUITE 800, WASHINGTON, DC, 20006-1021, US

(21) Appl. No.: 11/209,646

(22) Filed: Aug. 24, 2005

(30) Foreign Application Priority Data
Feb. 25, 2005 (JP) 2005-052005

(51) Int. Cl. G01N 33/537; G01N 33/543

(52) U.S. Cl. 435/7.92; 436/518

(57) ABSTRACT

The present invention aims at providing a method for measuring spinorphin in a sample collected from a living body accurately with high sensitivity. The present invention provides a method for measuring spinorphin in a sample collected from a living body, comprising the steps of: (A) mixing a sample collected from a living body with trichloroacetic acid to give a solution phase and subjecting said solution phase and a solvent to a reverse phase column to elute spinorphin with said solvent; (B) contacting the spinorphin eluted in the step (A) with spinorphin immobilized on a carrier and a spinorphin antibody; (C) removing the spinorphin antibody that has not been bound to the spinorphin immobilized on the carrier; (D) contacting the spinorphin antibody bound to the spinorphin immobilized on the carrier obtained in the step (C) with a labeled secondary antibody that specifically binds to said spinorphin antibody so as to bind the spinorphin antibody bound to the spinorphin immobilized on the carrier and the labeled secondary antibody; and (E) measuring the amount of label in the labeled secondary antibody bound to the spinorphin antibody.

### BACKGROUND OF THE INVENTION

1. Field of the Invention

The present invention relates to a method for measuring spinorphin in a sample separated from a subject. Furthermore, the present invention relates to use of said method for measuring spinorphin for the estimation of rheumatoid arthritis disease.

2. Description of the Related Art

Spinorphin is an endogenous physiologically active peptide consisting of seven amino acids, which is isolated from spinal cord and purified. Spinorphin is a compound having activities for inhibiting enkephalin-degrading enzymes which are involved in transmission and control of pain, and for inhibiting angiotensin converting enzyme (see JP-A-1991-167199). In recent years, spinorphin is considered to participate in various functions in immunization and inflammation-nervous system, such as inhibition of opioid activity, migrating ability of neutrophil and the like. Furthermore, spinorphin has a longer half-life in vivo than that of enkephalin. Therefore, spinorphin can provide better analgesic effect when it is administered in combination with enkephalin. Moreover, spinorphin passes a blood-brain barrier where it is externally administered to a living body. Therefore, spinorphin attracts attention as a substance that can act as a nonnarcotic analgesic agent that provides no side effect in a living body as an inhibitor of enkephalin-degrading enzymes (see Kinya Nishimura and other three authors, The Japanese journal of Anesthesiology (Masui), 1993, Vol. 42, pp. 1663-1670). However, the relationship between spinorphin and rheumatoid arthritis or osteoarthritis has not been known.

As a method for measuring spinorphin in a body fluid such as spinal cord fluid and the like, there is known a method comprising the development by high performance liquid chromatography (HPLC) using an octadecyl silica (ODS) column under linear concentration gradient (10 to 40%/20 min, 1.5 mL/min) of triacetylamine-phosphate buffer to acetonitrile (see JP-A-2000-95794).

However, in the case where multiple samples are measured simultaneously, this method requires long time. Furthermore, precise measurement of the concentration of spinorphin in the sample is difficult due to the degradation and the like of spinorphin in the sample. Therefore, this method is not sufficient.

### SUMMARY OF THE INVENTION

The present invention aims at providing a method for measuring spinorphin in a sample collected from a living body accurately with high sensitivity. Furthermore, the present invention aims at providing use of said method for the measurement of spinorphin for differentiating rheumatoid arthritis (hereinafter abbreviated as RA) from osteoarthritis (hereinafter abbreviated as OA) to readily estimate RA, which allows the diagnosis of RA.

The present inventors have done intensive studies on amethod for measuring spinorphin accurately in a sample collected from a living body with high sensitivity. As a result, the inventors have found that spinorphin in a sample of living body can be measured accurately with high sensitivity by treating a sample collected from a living body comprising spinorphin with trichloroacetic acid; filling the solution phase in a reverse phase column; eluting spinorphin with a solvent; and applying a specific competitive enzyme immunization method to the eluted spinorphin. Furthermore, the inventors have measured the amounts of spinorphin in the synovial fluid collected from an RA patient and an OA patient using said method for the measurement of spinorphin, and found that the amount of spinorphin in the synovial fluid from an RA patient is lower than that of an OA patient, which allows discrimination against RA patients. Based on these findings, the present inventors have done further studies, which resulted in the completion of the present invention.

Namely, the present invention relates to:
(1) a method for measuring spinorphin in a sample collected from a living body, comprising the steps of:
   (A) mixing a sample collected from a living body with trichloroacetic acid to give a solution phase and subjecting said solution phase to a reverse phase column to elute spinorphin with a solvent;
   (B) contacting the spinorphin eluted in the step (A) with spinorphin immobilized on a carrier and a spinorphin antibody;
   (C) removing the spinorphin antibody that is not bound to the spinorphin immobilized on the carrier;
   (D) contacting the spinorphin antibody bound to the spinorphin immobilized on the carrier obtained in the step (C) with a labeled secondary antibody that specifically binds to said spinorphin antibody so as to bind the spinorphin antibody bound to the spinorphin immobilized on the carrier and the labeled secondary antibody; and
   (E) measuring the amount of label in the labeled secondary antibody bound to the spinorphin antibody;
(2) use of the method for measuring spinorphin in a sample collected from a living body as described in the above (1), for the estimation of rheumatoid arthritis disease;
(3) the use as described in the above (2), wherein the sample is joint synovial fluid;
(4) the use as described in the above (3), wherein the measured concentration of spinorphin in the sample is not more than 5 ng/mL;
(5) a kit for the measurement of the concentration of spinorphin in a sample, comprising a spinorphin antibody, a spinorphin to be immobilized on a carrier, a carrier for immobilizing spinorphin, and a labeled secondary antibody;
(6) a kit for the measurement of the concentration of spinorphin in a sample, comprising a spinorphin antibody, a carrier on which spinorphin is immobilized, and a labeled secondary antibody; and
(7) the kit as described in the above (5) or (6), wherein the carrier is a microtiter plate.

According to the present invention, spinorphin can be measured accurately with high sensitivity by pre-treating a sample using a reverse phase column and subjecting the treated sample to a specific competitive enzyme immunization method.

According to the present invention, spinorphin in multiple samples can be measured, within a time period in which changes of concentration due to the degradation and the like of spinorphin in the samples are negligible.

In the case where the amount of spinorphin in the synovial fluid collected from a joint of a patient suffering from joint pain is measured to be not more than 5 ng/mL according to the method for the measurement of spinorphin of the present invention, the patient can be estimated to be an RA patient.

### BRIEF DESCRIPTION OF THE DRAWING

FIG. 1 shows a FIGURE indicating the time-dependent change of the relative activity of the concentration of spinorphin in bovine cerebrospinal fluid.

### DETAILED DESCRIPTION OF THE PREFERRED EMBODIMENTS

Spinorphin is an object of the measurement method of the present invention. Spinorphin is an oligopeptide having the amino acid sequence consisting of seven amino acid residues represented by sequence listing: SEQ ID NO: 1

Leu-Val-Val-Tyr-Pro-Trp-Thr and is soluble in water and organic solvents. Spinorphin was first found in bovine cerebral spinal cord as an in vivo inhibitor of an enkephalin-degrading enzyme. Spinorphin can be prepared by a known method, e.g., a method described in JP-A-2000-95794.

The sample that can be used in the present invention is not specifically limited, and any sample comprising spinorphin can be used. Examples of the sample may include samples from a living body such as cerebrospinal fluid, joint synovial fluid, blood, plasma, serum, saliva and urine, and organs, tissues and the like collected from animals such as humans, rats, mice, dogs, cattle, cats, rabbits, guinea pigs and the like.

Hereinafter the steps in the method of the present invention are explained.

Firstly, in the step (A), a sample collected from a living body is mixed with trichloroacetic acid to give a solution phase, and said solution phase is then subjected to a reverse phase column with a solvent to elute spinorphin.

Although the usage form of trichloroacetic acid varies depending on the kind of the sample collected from a living body to be measured, it is preferable to use an aqueous solution of trichloroacetic acid comprising trichloroacetic acid of about 5 to 20% by mass. The amount of trichloroacetic acid is preferably an amount sufficient to precipitate proteins in the sample collected from a living body. It is preferable to add the aqueous solution of trichloroacetic acid in an amount of about 0.5 to 2 volume ratio relative to 1 volume of the solution of the sample collected from a living body. The solution phase can be separated by a known method such as filtration, centrifugation or the like.

Preferable examples of the reverse phase column used for the present invention may include an ODS column and the like. The ODS column is a column filled with a filler in which octadecylsilyl groups (ODS groups, C18 groups) have been chemically bound to a silica gel carrier. Examples of the ODS column may include ODS-A 60-60/30 (manufactured by YMC Co., Ltd.), Inertsil ODS (manufactured by GL Sciences, Inc.), L-column ODS (manufactured by Chemicals Evaluation and Research Institute, Japan), Develosil ODS UG-5 (manufactured by Nomura Chemical Co., Ltd.), CAPCELL PAK C18 MGII (manufactured by Shiseido Co., Ltd.), ZORBAX XDB-C18 (manufactured by Yokogawa Analytical Systems Inc.), Symmetry C18 (manufactured by Waters, Inc.), Nucleosil C18 (manufactured by M. Nagel Co., Inc.) and the like.

Examples of the solvent to be used to elute spinorphin may include alcohols having 1 to 5 carbon atom(s) (e.g., methanol, ethanol, propanol, and the like) and acetonitrile, a mixed solvent of two or more kinds of these solvents, and a mixed solvent of the above-mentioned solvent and water.

Secondly, in the step (B), the spinorphin eluted in the step (A) is contacted with spinorphin immobilized on a carrier and a spinorphin antibody.

Examples of the carrier on which spinorphin is to be immobilized may include a microtiter plate, a test tube, polymer beads and the like. Since spinorphin is an oligopeptide, a carrier having active functional groups such as amino groups, carboxyl groups and the like on the surface, and a carrier having a coat slide of a hydrophobic polymer [e.g., Immuno Plate, MaxiSorp (Nalge Nunc International)] and the like are preferred. It is preferable that spinorphin is immobilized on a carrier by covalent bonding of spinorphin to the surface of the carrier. It is preferable that said bonding is carried out by dissolving spinorphin in a buffer such as phosphate buffer, Tris-HCl saline or the like, contacting said solution with the carrier, and incubating the carrier at a temperature of about 0 to 50° C., preferably at room temperature, for at least not less than about 30minutes, preferably about 30 to 120 minutes. It is preferable that the carrier on which spinorphin has been immobilized is blocked with a blocking agent. Examples of the blocking agent may include skim milk, Block Ace (trade mark) and the like.

As a preferable spinorphin antibody used for the present invention, any antibody can be used so long as it specifically binds to spinorphin. The spinorphin antibody can be prepared easily by sensitizing an animal (e.g., rabbit, rat or the like) using spinorphin as an antigen, and separating and purifying the obtained antibody by the conventional method. It is preferable to conjugate spinorphin with a carrier protein so as to increase immunogenicity, because spinorphin is a peptide consisting of seven amino acids and is generally too short to exhibit immunogenicity. Examples of such carrier protein for conjugation may include, for example, KLH (Keyhole Limpet Hemocyanin), bovine serum albumin, ovalbumin and the like. Examples of the method for conjugation may include a method comprising activating the C-terminal carboxyl group of spinorphin with a carbodiimide (e.g., 1-ethyl-3-(3-dimethylaminopropyl)carbodiimide, etc.) and reacting the activated spinorphin with a primary amine of a carrier protein (EDC method), a method comprising bonding an amino group of a protein and an SH group of a peptide using MBS (maleimide benzoyloxysuccinimide) type crosslinking agent (MBS method) and the like.

As the antibody prepared by the above-mentioned method, either a polyclonal antibody or a monoclonal antibody can be used.

It is preferable that the spinorphin antibody obtained as above is specific to spinorphin. It is preferable that the spinorphin antibody does not cross-react with spinorphin analogues such as VVYPWT (sequence listing: SEQ ID NO: 2), VYPWT (sequence listing: SEQ ID NO: 3), hemorphin-4 (YPWT; sequence listing: SEQ ID NO: 4), PWT, LVVYPW (sequence listing: SEQ ID NO: 5), tynorphin (VVYPW; sequence listing: SEQ ID NO: 6), VYPW (sequence listing: SEQ ID NO: 7), YPW, LVVYP (sequence listing: SEQ ID NO: 8), WYP (sequence listing: SEQ ID NO: 9), VYP and the like.

It is preferable that the contact of the spinorphin eluted in the step (A) with the spinorphin immobilized on the carrier and the spinorphin antibody is performed by contacting a solution in which the above-mentioned rabbit spinorphin antibody and the spinorphin eluted in the step (A) have been dissolved in a buffer (e.g., Tris buffered saline, phosphate buffer, etc.), which is optionally diluted, with a carrier on which spinorphin has been immobilized. It is preferable that said contact is carried out usually at 0° C. to room temperature for about 30 minutes to 2 hours, preferably about 30 minutes to 1 hour, while allowing to stand or shaking slowly. During said reaction, the spinorphin immobilized on a carrier and the spinorphin eluted in the step (A) compete with the spinorphin antibody so that the spinorphin immobilized on a carrier can bind to the spinorphin antibody.

Subsequently, in the step (C), the spinorphin antibody that is not bound to the spinorphin immobilized on a carrier is removed.

It is preferable that said removal is carried out, for example, by decanting the carrier to discard the solution contacting with the carrier, or aspirating the solution using a pipette. It is preferable that the carrier is further washed with the above-mentioned buffer and the like at least 3 times, preferably about 3 to 6 times.

Then, in the step (D), the spinorphin antibody bound to the spinorphin immobilized on the carrier obtained in the step (C) is contacted with a labeled secondary antibody that specifically binds to said spinorphin antibody so as to bind the spinorphin antibody bound to the spinorphin immobilized on the carrier and the labeled secondary antibody.

Examples of the labeled secondary antibody may include anti-rabbit IgG goat IgG antibody, goat anti-mouse antibody and the like that are labeled with a label enzyme or a fluorescence dye. Examples of the label enzyme may include peroxidase, glucose oxidase, acidic phosphatase, alkaline phosphatase and the like, preferably peroxidase, and especially preferably horseradish peroxidase. Examples of the labeling fluorescence dye may include MFP 488, Alexa Fluor 488, rhodamine, fluoresceine, Cy2, Cy3 and the like.

The binding of the spinorphin antibody bound to the spinorphin immobilized on a carrier with the labeled secondary antibody is preferably carried out generally at 0° C. to room temperature, while allowing to stand or shaking slowly, for about 30 minutes to 2 hours, preferably about 30 minutes to 1 hour.

In the step (E), the amount of label in the labeled secondary antibody bound to the spinorphin antibody in the step (D) is measured.

In the case where a secondary antibody labeledwith an enzyme, for example, is used, the amount of label in the labeled secondary antibody bound to the spinorphin antibody bound to the immobilized antigen (spinorphin) can be measured by measuring the light absorbance at the wavelength corresponding to the color tone of the color reaction between the label enzyme and the substrate. The color reaction between the label enzyme and the substrate can be carried out by contacting the substrate solution with the enzyme-labeled secondary antibody. The substrate differs depending on the label enzyme to be used. In the case where the label enzyme is, for example, horseradish peroxidase, examples of the preferable substrate to be used may include o-phenylenediamine, 3,3′,5,5′-tetramethylbenzidine and the like. It is preferable that the light absorbance is measured within about 10 minutes to 60 minutes after coloring.

In the case where fluorescence labeling is used, the fluorescence intensity may be measured using a fluorometer.

In the case where the carrier is amicroplate, it is preferable to use an autoreader or a microplate reader that can measure light absorbance successively and automatically in the casewhere enzyme labeling is used, and it is preferable to use a fluorescence microreader or the like in the case where fluorescence labeling is used.

In the above-mentioned method according to the present invention, spinorphin can be measured quantitatively in the concentration range of about 10⁻¹⁰ to 10⁻⁷ g/mL. Accordingly, in the case where a sample comprising spinorphin at high concentration is used, it is preferable to dilute the sample appropriately with the above-mentioned buffer and the like.

By using the method for the measurement of the present invention, in the case where the amount of spinorphin in the synovial fluid collected from the joint of a subject is measured to be about not more than 5 ng/mL, the subject can be estimated to be suffering from rheumatoid arthritis. Therefore, said method is useful for the diagnosis of rheumatoid arthritis.

Furthermore, the present invention relates to a kit for the measurement of the concentration of spinorphin in a sample, which comprises a spinorphin antibody, an antigen for coating (spinorphin), a microtiter plate and a labeled secondary antibody.

For the kit for the measurement of the concentration of spinorphin of the present invention, examples of the spinorphin antibody may include a spinorphin rabbit antibody and the like. Examples of the antigen for coating may include a solution of spinorphin in a buffer. Examples of the preferable microtiter plate may include a microtiter plate having active functional groups such as amino groups, carboxyl groups and the like on the surface, a microtiter plate having a coat slide of a hydrophobic polymer. Alternatively, a kit comprising a microtiter plate on which spinorphin as an antigen has been immobilized, a spinorphin antibody and a labeled secondary antibody instead of an antigen for coating and a microtiter plate may be used. Examples of the preferable labeled secondary antibody may include the above-mentioned enzyme-labeled secondary antibody and fluorescence-labeled secondary antibody. In the case where an enzyme-labeled secondary antibody is used for a kit, it is preferable that the above-mentioned substrate and the like are also included in the kit. Furthermore, it is preferable that other reagents such as a washing liquid for plate [a buffer comprising a surfactant (e.g., Polysorbate 20, etc.) and the like], a buffer (e.g., phosphate buffer, Tris buffer saline) and the like are included in the kit.

According to the kit for the measurement of the concentration of spinorphin of the present invention, the concentration of spinorphin in a sample can be measured easily and accurately.

### EXAMPLES

Hereinafter the present invention is explained with referring to Examples, but the present invention is not limited thereto.

### Example 1

(1) Production of a Spinorphin Rabbit Antibody

Spinorphin (obtained from American Peptide Company Inc., Sunnyvale, Calif., USA) was bound to KLH (keyhole limpet hemocyanin) using EDC (1-ethyl-3-(3-dimethylaminopropyl)carbodiimide) to give a KLH-bound spinorphin. The KLH-bound spinorphin was injected subcutaneously to the backs of two rabbits (New Zealand white). The KLH-bound spinorphin was immunized 8 times at the invervals of 2 weeks. After the last immunization was completed, blood was collected from the heart of the rabbit. Serum was separated from the collected whole blood to give a spinorphin rabbit antibody. The titer of the antibody was measured by reacting a solid antigen (200 ng/well) with diluted serum (100 μL/well), washing, adding the secondary antibody, adding the substrate and measuring the coloring. The serum was diluted firstly 1000-fold, then every 3-fold and at the maximum 81000-fold to prepare a diluted serum, and said diluted serum was used for the reaction.

(2) Enzyme Immunoassay

A sample containing spinorphin (2 mL) was mixed with an equivalent amount of trichloroacetic acid (10% by mass), and the mixture was allowed to stand at 4° C. for 1 hour. The mixture was centrifuged at 1500 xg for 20 minutes, and the supernatant was injected onto an ODS column (ODS-A 60-60/30; manufactured by YMC Co., Ltd.). The column was washed with 10-fold volume of water, and spinorphin was eluted with 80% (v/v) aqueous methanol solution. The solvent was evaporated, and the eluted substance was dissolved in Tris buffer saline (TBS, 0.5 mL) and served as a sample.

Spinorphin (50 ng) and TBS (100 μL) were placed in a 96-well plate (Nunc-Immuno Plate, MaxiSorp Surface, Nalge Nunc International, Denmark), and the reaction was carried out at room temperature for 1 hour to immobilize spinorphin on the plate. The plate on which spinorphin had been immobilized was washed with TBS comprising 0.1% by mass of Tween 20 (TBS-T, 10 mM) 5 times. The plate on which spinorphin had been immobilized was then blocked with TBS-T comprising skim milk (10% by mass) and sodiumazide (0.1% by mass) at room temperature for 1 hour. Skim milk was removed from the plate by washing. To the plate were added a diluted solution of the sample (100 μL) and the spinorphin rabbit antibody (5 μg/well), and the plate was subjected to competitive reaction. The reaction was carried out for 1 hour by shaking the plate slowly. The plate was then washed 5 times, and horseradish peroxidase (HRP)-labeled F (ab′)₂ goat anti-rabbit IgG (H+L) (100 μL, diluted by 1000-fold) was added to the plate and reacted at room temperature for 1 hour. After the reaction was completed, the plate was washed 5 times, and a citric acid buffer comprising o-phenylenediamine (2.2 μM) and 0.014% (v/v) aqueous hydrogen peroxide (0.1 M, pH 5, 100 μL) was then added to the plate. The plate was allowed to stand for 20 minutes, and the light absorbance of the solution in the wells on the plate was measured at 405 nm using an autoreader (Korona Electric Co. Ltd., Ibaragi, Japan). Under these conditions, the reactivity of the antibody was about 1.2 to 1.5 O.D. (optical density). The concentration of spinorphin in each sample was calculated by specifying %B/Bo (light absorbance ratio; value obtained by dividing light absorbance with light absorbance at blank 0) of the standard curve.

(3) Preparation of a Standard Curve

A calibration curve was prepared according to the following procedures.

Preparation of a standard solution of spinorphin: A standard solution of spinorphin was prepared by dissolving spinorphin in TBS so that the concentration of spinorphin became 10⁻¹² to 10⁻⁶ g/mL.

Preparation of a standard curve: A standard curve was prepared according to a similar manner to the above-mentioned (2), except that a standard solution of spinorphin (each 100 μL) was added instead of the diluted solution of the sample (100 μL) used in the above-mentioned (2).

(4) Variation of Spinorphin Concentration in a Sample over Measurement Time

A test was carried out to confirm whether the activity of spinorphin could be retained through the period from the step (B) to the step (E). Spinorphin (1 μg/mL) in TBS was dissolved in bovine cerebrospinal fluid. The cerebrospinal fluid was allowed to stand at room temperature for various time periods. The reaction was stopped, and the amount of spinorphin was measured according to the method mentioned in the above (2).

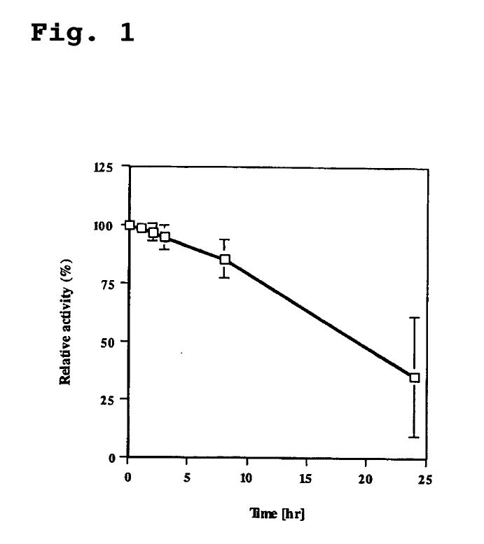

The result is shown in FIG. 1. The relative activity of the amount of spinorphin gradually decreased to 98.8±0.98% after allowing to stand for 1 hour and decreased to 35.4±26.1% (n=3) after 24 hours relative to the amount of spinorphin added to bovine cerebrospinal fluid. The decomposition of spinorphin during 1 hour, which is the time period required for the competitive reaction in the method of the present invention, was estimated to only 1.2%, and said value was not a significant difference in a static significant difference test. Accordingly, it is shown that the amount of spinorphin in a sample can be measured effectively and accurately by the method for the measurement of spinorphin according to the present invention.

(5) Cross Reactivity of Spinorphin Analogues

Furthermore, the method of the above-mentioned (2) was carried out using spinorphin analogues VVYPWT (sequence listing: SEQ ID NO: 2), VYPWT (sequence listing: SEQ ID NO: 3), hemorphin-4 (sequence listing: SEQ ID NO: 4), PWT, LVVYPW (sequence listing: SEQ ID NO: 5), tynorphin (sequence listing: SEQ ID NO: 6), VYPW (sequence listing: SEQ ID NO: 7), YPW, LVVYP (sequence listing: SEQ ID NO: 8), WYP (sequence listing: SEQ ID NO: 9) and VYP. The above-mentioned spinorphin analogues were synthesized according to a method described in JP-A-2000-95794. The results are shown in Table 1. The cross reactivities of VVYPWT and VYPWT were each not more than 0.0004-fold of that of spinorphin and were neglectable. Other spinorphin analogues (hemorphin-4, PWT, LVVYPW, tynorphin, VYPW, YPW, LVVYP, VVYP and VYP) could not be detected according to the method of the present invention. It was confirmed that the spinorphin analogues as competitive compounds for the spinorphin immobilized on the plate did not react with the spinorphin antibody. This fact shows that the method for the measurement of spinorphin according to the present invention can measure spinorphin specifically and quantitatively.

TABLE 1

| | | Cross |
|---|---|---|
| Compound | IC₅₀ (g/mL) | reactivity¹⁾ |
| Spinorphin | 6.4 × 10⁻¹⁰ ± 6.1 × 10⁻¹⁰ | 1 |
| VVYPWT | 1.6 × 10⁻⁶ ± 5 × 10⁻⁶ | 0.0004 |
| VYPWT | >1.0 × 10⁻⁵ | <6.4 × 10⁻⁵ |

¹⁾Cross reactivity is shown as a ratio of spinorphin relative to IC₅₀.

### EXAMPLE 2

Measurement of Spinorphin in the Synovial Fluid from a Rheumatoid Arthritis (RA) Patient and an Osteoarthritis (OA) Patient

(1) Collection of Samples

Synovial fluid was collected from OA patients (40 persons) and RA patients (39 persons). A sample of synovial fluid was sucked from the knee joint of an outpatient, placed into a plastic container and centrifuged at 4° C. for 10 minutes at 1500 xg. The supernatant was kept at −20° C. until analysis. Diagnosis of OA and RA was based on the “ACR Revised Diagnosis Criteria” by the American College of Rheumatology (ACR). The ages of OA patients were 74 to 88 years old (11 men and 29 women), and their average morbid period was 6 years (range: 0.1 to 20 years). The ages of RA patients were 44 to 74 years old (6 men, 32 women), and their average morbid period was 14.5 years (range: 1 to 36 years, least erosive subset (LES) 3 persons, more erosive subset (MES) 29 persons, multilating disease (MUD) 6 persons). All of the RA patients took several kinds of medicaments. The medicaments were an anti-inflammatory agent, a gold compound, methotrexate, sulfasalazine, a corticosteroid, bucillamine and D-penicillamine. There was no person who had received a corticosteroid or an intra-articular steroid at high concentration before sucking of the synovial fluid. The approval from each patient was reviewed by the Committee, and individual informed concept was obtained from each patient.

(2) Measurement of Spinorphin in the Synovial Fluid

The amounts of spinorphin in the synovial fluid collected from the joints of RA patients and OA patients were measured according to the method of the above-mentioned (1). As aresult, the amounts of spinorphin in the synovial fluid collected from the joints of RA patients were 4.2±3.4 ng/mL (n=20), and those of OA patients were 10.1±7.1 ng/mL (n=23). The amounts of spinorphin in RA patients were significantly lower than those of OA patients (P<0.01). This fact suggests that in the case where the amount of spinorphin in the synovial fluid from the joint of a patient is not more than 5 ng/mL, the patient can be estimated to be an RA patient rather than an OA patient.

### REFERENCE EXAMPLE

Synthesis of Spinorphin Analogues (PWT and YPWT)

(1) Synthesis of PWT

Boc-Thr-Merrifield resin (1 equivalent amount) wherein Boc-Thr-OH (the third protective amino acid of PWT) was bound to the resin was placed in a reaction vessel, washed with DCM (dichloromethane) and swelled repeatedly. The resin was contacted with a deblock solution (containing 50% (v/v) TFA (trifluoroacetic acid)/DCM) for 30 minutes to remove Boc (t-butoxycarbonyl) group. The residual TFA was removed with isopropanol, and the resin was washed with DCM. The resin was neutralized with DCM comprising 5% (v/v) of diisopropylethyleneamine, and washed with DCM. The resin was then reacted in a small amount of a mixed solution of 4 equivalent amounts of Boc-Trp-OH (the second protective amino acid of PWT) and 4 equivalent amounts of DCC (dicyclohexylcarbodiimide) for 2 hours at room temperature. The resin was washed successively with DMF and DCM to give Boc-Trp-Thr-Merrifield resin. Similarly, amino acid was coupled using a protective amino acid Boc-Pro-OH to give Boc-Pro-Trp-Thr-Merrifield resin. The obtained Boc-Pro-Trp-Thr-Merrifield resin was reacted in anhydrous hydrogen fluoride comprising anisole (10% by mass) and 1,2-ethanedithiol (5% by mass) at 0° C. for 1 hour. Hydrogen fluoride was removed by evaporation, and the resin was washed with ether. Pro-Trp-Thr was extracted from a mixture of the obtained peptide (Pro-Trp-Thr) and the resin using 10% (v/v) acetic acid, and lyophilized to give crude Pro-Trp-Thr. The crude Pro-Trp-Thr was dissolved in 0.1% (v/v) TFA solution, developed by high performance liquid chromatography (using an octadecyl silica (ODS) column; linear concentration gradient (10 to 50% (v/v)/20 min) of acetonitrile containing 0.1% (v/v) of TFA) to elute a fraction corresponding to Pro-Trp-Thr from the column, which was then lyophilized to give PWT.

(2) Synthesis of Hemorphin-4 (YPWT)

Boc-Tyr(BrZ)-Pro-Trp-Thr-Merrifield resin was obtained according to a similar method to the above-mentioned (1) by coupling a protective amino acid Boc-Tyr(BrZ)-OH to Boc-Pro-Trp-Thr-Merrifield resin obtained in the above-mentioned (1). The peptide was extracted and purified according to a similar method to the above-mentioned (1) to give hemorphin-4 (YPWT; sequence listing: SEQ ID NO: 4). As used herein, BrZ is a brominated benzyloxycarbonyl protective group.

VYPWT and VVYPWT can be produced similarly by successively coupling a protective amino acid Boc-Val-OH to Boc-Tyr(BrZ)-Pro-Trp-Thr-Merrifield resin. Furthermore, other spinorphin analogues LVVYPW, tynorphin (VVYPW) , VYPW, YPW, LVVYP, VVYP and VYP can be similarly produced.

### INDUSTRIAL APPLICABILITY

The present invention is useful for measuring the concentration of spinorphin in a sample within the range of the concentration of spinorphin from 10⁻¹⁰ to 10⁻⁷ g/mL. In addition, such measurement method of the present invention can be used for the diagnosis of an RA patient.

1. A method for measuring spinorphin in a sample collected from a living body, comprising the steps of:
(A) mixing a sample collected from a living body with trichloroacetic acid to give a solution phase and subjecting said solution phase to a reverse phase column to elute spinorphin with a solvent;
(B) contacting the spinorphin eluted in the step (A) with spinorphin immobilized on a carrier and a spinorphin antibody;
(C) removing the spinorphin antibody that is not bound to the spinorphin immobilized on the carrier;
(D) contacting the spinorphin antibody bound to the spinorphin immobilized on the carrier obtained in the step (C) with a labeled secondary antibody that specifically binds to said spinorphin antibody so as to bind the spinorphin antibody bound to the spinorphin immobilized on the carrier and the labeled secondary antibody; and
(E) measuring the amount of label in the labeled secondary antibody bound to the spinorphin antibody.

2. A method for the estimation of rheumatoid arthritis disease, which comprises the steps of:
(A) mixing a sample collected from a living body with trichloroacetic acid to give a solution phase and subjecting said solution phase to a reverse phase column to elute spinorphin with a solvent;
(B) contacting the spinorphin eluted in the step (A) with spinorphin immobilized on a carrier and a spinorphin antibody:
(C) removing the spinorphin antibody that is not bound to the spinorphin immobilized on the carrier;
(D) contacting the spinorphin antibody bound to the spinorphin immobilized on the carrier obtained in the step (C) with a labeled secondary antibody that specifically binds to said spinorphin antibody so as to bind the spinorphin antibody bound to the spinorphin immobilized on the carrier and the labeled secondary antibody; and
(E) measuring the amount of label in the labeled secondary antibody bound to the spinorphin antibody.

3. The method as claimed in claim 2, wherein the sample is joint synovial fluid.

4. The method as claimed in claim 3, wherein the measured concentration of spinorphin in the sample is not more than 5 ng/mL.

5. A kit for the measurement of the concentration of spinorphin in a sample, comprising a spinorphin antibody, a spinorphin to be immobilized on a carrier, a carrier for immobilizing spinorphin, and a labeled secondary antibody.

6. A kit for the measurement of the concentration of spinorphin in a sample, comprising a spinorphin antibody, a carrier on which spinorphin is immobilized, and a labeled secondary antibody.

7. The kit as claimed in claim 5 or 6, wherein the carrier is a microtiter plate.

8. The kit as claimed in claim 6, wherein the carrier is a microtiter plate.


SEQUENCE LISTING (9 sequences)

SEQ ID NO: 1 - protein, 7 aa, Artificial Sequence
  Description of Artificial Sequence Synthetic Peptide
LVVYPWT

SEQ ID NO: 2 - protein, 6 aa, Artificial Sequence
  Description of Artificial Sequence Synthetic Peptide
VVYPWT

SEQ ID NO: 3 - protein, 5 aa, Artificial Sequence
  Description of Artificial Sequence Synthetic Peptide
VYPWT

SEQ ID NO: 4 - protein, 4 aa, Artificial Sequence
  Description of Artificial Sequence Synthetic Peptide
YPWT

SEQ ID NO: 5 - protein, 6 aa, Artificial Sequence
  Description of Artificial Sequence Synthetic Peptide
LVVYPW

SEQ ID NO: 6 - protein, 5 aa, Artificial Sequence
  Description of Artificial Sequence Synthetic Peptide
VVYPW

SEQ ID NO: 7 - protein, 4 aa, Artificial Sequence
  Description of Artificial Sequence Synthetic Peptide
VYPW

SEQ ID NO: 8 - protein, 5 aa, Artificial Sequence
  Description of Artificial Sequence Synthetic Peptide
LVVYP

SEQ ID NO: 9 - protein, 4 aa, Artificial Sequence
  Description of Artificial Sequence Synthetic Peptide
VVYP